(19) United States
(12) Patent Application Publication
(10) Pub. No.: US 2023/0173297 A1
(43) Pub. Date: Jun. 8, 2023

(54) BLACK LIGHT DEVICE FOR IMPROVING VITAMIN D3 FORMATION IN ANIMALS AND INACTIVATING BACTERIA AND VIRUSES

(71) Applicant: FARMER-LIGHT HOLDING A/S., Herning (DK)

(72) Inventor: Povl KAAS, Herning (DK)

(21) Appl. No.: 17/997,951

(22) PCT Filed: Feb. 4, 2021
(86) PCT No.: PCT/EP2021/052653

§ 371 Date: Nov. 4, 2022

(30) Foreign Application Priority Data
May 7, 2020 (EP) 20173423.3
Nov. 24, 2020 (EP) PCT/EP2020/083177

(51) Int. Cl. A61N 5/06

(52) U.S. Cl. CPC A61N 5/0616; A61N 5/0625; A61N 2005/0659; A61N 2005/0661

(57) ABSTRACT

The present disclosure relates to light emitting devices, systems and methods for use in animal nests, such as mink nests or piglet nests, for enhancing the formation of ND3 in animals and minimizing the microbial pressure in the animal nest, while providing the animals with an improved resting condition. A light emitting device comprises at least one UVB light source, wherein the light emitting device is configured such that UV light below 285 nm is not emitted from the device, visible light between 380 and 750 nm is not emitted from the device, and light in a wavelength interval of 290 nm-315 nm is emitted from the device.

The invention relates to a light emitting device, for formation of natural vitamin D3 (ND3), in particular in a mammal, more particular a young or newborn mammal, such as a piglet or a mink kit, and provision of heat to the animal, without emitting any visible light, a method for improving the formation of ND3 in an animal and inactivating bacteria and viruses.

### BACKGROUND

Natural light enhances the natural production of vitamin D3 in the skin of humans and animals. Vitamin D3 is produced in the skin from 7-dehydrocholesterol by ultra violet (UV) light of the B-type (UVB). UVB is present in the spectrum of natural light and hence the exposure of skin to natural light drives the formation of ND3 in the skin. ND3 has a crucial function for the immune system and in the development and maintenance of the skeleton and bones.

Domestic animals often spend a large fraction or even all of their time indoors, with piglets and mink kits spending most of their time in the nests. For example, a suckling pig is only outside the nest approximately 16 times a day to suck for approximately 15 min, equivalent to only 4 hours per 24-hour day. The same conditions apply to mink kits. This has resulted in a weakened immune system for the animals due to the lack of natural light necessary for the animals to ensure the natural production of ND3.

The conditions in typical animal nest facilities, such as mink kit nest facilities or piglet nest facilities, may promote the growth of a wide diversity of microorganisms including bacteria and virus, including coronavirus. Presence of airborne microorganisms may affect the quality of air in the facilities and the neighboring areas. The combination of a poor air quality and a weakened immune system makes the animals at risk of contracting diseases.

In order to ensure that the animals get a sufficient amount of vitamin D3 various approaches have been used. One approach is to feed the animal a synthetic vitamin D3 (SD3) in the form of dietary supplement added to the animal feed. One example is the enriching of animal food with SD3 or by adding a pill or a powder of SD3.

Even though SD3 and ND3 are chemically identical they function differently in the body of an animal. SD3 does not bind to the proper transport proteins, in the manner that ND3 does, but instead SD3 remains in the residual fat of the blood after it has been absorbed through the intestinal tract. This is considered crucial for the biological effect of the vitamin and explains why large doses of SD3 are toxic whereas ND3 cannot be overdosed.

New-born mink kits and new-born piglets are young mammalian animals that are very sensitive to the lack of vitamin ND3 as they are born with no measurable level of ND3 in their blood, and hence have a weakened immune system.

Mink kits are born with a very immature immune system and with low serum concentrations of circulating immunoglobulins, similar to new-born piglets. Therefore, it is crucial that these animals, soon after birth, reach high blood concentrations of IgG through passive immunization by receival of IgG from the mother through colostrum (raw milk).

In the same way, new-born piglets are dependent on receiving ND3 through the breastmilk from the sow. However, the content of ND3 in the breastmilk is very low. In nature this is not a problem as the feral pigs give birth to their piglets in the summer in which the need for ND3 is entirely covered by the UVB radiation from the sun. The lack of ND3 plays a key role in the domesticated piglets' ability to fight infections and hence to the mortality of piglets in conventional production. The use of UV light to enhance the formation of ND3 is described in EP 2558984.

### SUMMARY

Unfortunately, the illumination strategy is challenging at best, one reason being that the intensity of the light sources is too low. While the illumination time is typically increased in order to solve this problem, the present inventor has realized that even small amounts of visible light stresses the animal and disturbs the sleep in such a way that it keeps the animal from falling into a deep sleep. This may have a significant effect on the well-being of the animal, such as a suckling pig, a piglet, or a mink kit, and it has been shown that an animal that is able to get a sufficient amount of deep sleep grows faster and is healthier than one that does not get a sufficient amount of deep sleep.

Another issue is that young animals require darkness and rest with a healthy body temperature. For suckling pigs and piglets this temperature is typically 39° C. to 40° C., and it is crucial for their survival. A healthy body temperature, for example in piglets is nowadays typically ensured by a standard IR lamp that not only emits IR radiation but also visible light, which is a source of disturbance. In a mink kit nest the optimal temperature range is between 7° C. and 25° C. and this is normally ensured by closed cubic nest with closed walls with a single hole in one of the walls for entrance and exit. The nest can be lined with e.g. straw such that an additional heat source is typically not necessary for mink kit nests, the lining and the construction of the mink nest can be provided such that the atmospheric conditions are optimal for the mink kits inside the mink kit nest.

The lighting conditions for formation of ND3, and consequently a healthy concentration of ND3 in the plasma, is one of the cornerstones for a strong immune system. Lighting conditions comprising visible light have been identified as leading to disturbances to the sleeping patterns of suckling pigs, piglets, and mink kits. This has shown to result in an impeded growth which is a factor that has shown a strong correlation with a higher mortality. Similarly, hypothermia is a large problem in animal facilities, such as piglet or mink facilities. In fact, hypothermia is the single most important cause of piglet mortality, for example pre-weaning piglet mortality. One embodiment of the present disclosure therefore relates to providing the animals, such as suckling pigs, piglets, or minks, with an optimal environment for relaxing and sleeping. Black light, and optionally IR, without visible light may ensure that the animals fall asleep quickly in a deep and calm sleep with a stable circadian rhythm. This allows the animals, such as suckling pigs, piglets, or minks to be able to wake up fully rested with a healthy core body temperature.

One object of the present invention is to provide a light emitting device suitable for enhancing ND3 formation in the skin of an animal, such as a new-born piglet or a new-born mink kit. The presently disclosed light emitting device serves a way to ensure the ND3 formation in the skin of the animal without the need of long-term exposure to visible light, which would otherwise stress the animal during its relaxation and sleep. The present disclosure therefore relates to a light emitting device for use in an animal nest for enhancing the formation of ND3 in animals in the animal nest.

A further object of the present invention is to provide a light emitting device suitable for reducing the microbial pressure in an animal farm production facility. The light emitting device is therefore preferably configured to emit light having a wavelength range that inactivates microbes, such as bacteria and viruses.

In an embodiment of the present disclosure the light emitting device comprises at least one light source, and the light emitting device is configured such that light in a wavelength interval of 280 nm-315 nm, more preferably 285 nm-315 nm, yet more preferably 290 nm-315 nm, even more preferably 290 nm-311 nm, most preferably 290 nm-305 nm, is emitted from the device. Preferably UV light below 280 nm, more preferably below 285 nm, most preferably below 290 nm, is not emitted from the device, as UVC light can harm the animals. And preferably visible light is not emitted from the device, such that light from the presently disclosed light emitting device appears as black light to the human eye and in particular to the animal eye, such as the piglet eye and/or the mink eye. In that regard it is noted that a light emitting device that emits visible light is not suitable and/or configured for use in an animal nest, in particular a piglet nest and a mink nest, because the visible light emitted from the device will disturb the young animals during sleep.

The present disclosure hence regards a device which can be used to illuminate animals with light in a wavelength interval of around 290 nm-315 nm, but without disturbing the animals by illumination with visible light. This is advantageous to ensure a calm, stress-free sleep of the animal, without the presence of visible light, while being illuminated with UVB light within the wavelength interval of 290 nm-315 nm from the light emitting device.

In a preferred embodiment of the present disclosure, the light emitting device is configured such that only a single lamp housing and/or lamp socket is required for accommodating said device. The light emitting device may thereby be a polychromatic lamp, configured to emit light with wavelengths and intensities as disclosed herein.

A further object of the present disclosure is to ensure that the animals, e.g. piglets and/or minks, are able to maintain a healthy core body temperature. Hypothermia has been identified as one of the major causes of piglet mortality. Studies have shown that body temperature in the first couple of hours after farrowing is crucial to early piglet survival, and still is of major importance during weaning at 4-5 weeks. The situation is similar for mink kits. Therefore, in one embodiment of the present disclosure light emitting device is emitting infrared radiation, such that the light emitting device is emitting light having wavelengths within the interval of 290 nm-315 nm, such as within the interval of 290 nm-305 nm, and light having wavelengths above 700 nm, more preferably light having wavelengths above 750 nm, in principle within the interval of 700 nm-1 mm, more preferably within the interval of 750 nm-1 mm, without emitting any visible light.

It is a strong preference that the light emitting device is configured such that the animals, e.g., piglets, are provided with an environment that promotes the build-up of a strong immune system and allows for good sleeping conditions, while at the same time ensuring that the animals maintains a healthy core body temperature, for example 39° C.-40° C. for piglets. Therefore, in one embodiment of the present disclosure, the light emitting device is configured to emit light having wavelengths within the interval of 290 nm-315 nm, and light having wavelengths above 700 nm, more preferably light having wavelengths above 750 nm, in principle within the interval of 700 nm-1 mm, more preferably within the interval of 750 nm-1 mm, without emitting any visible light. The addition of a IR light source is most relevant for piglets, because a mink nest typically does not need additional heat sources.

The presently disclosed light emitting device hence does not refer to a device emitting visible light, but rather a device emitting invisible light, in particular light in the UVB range, more specifically in the wavelength interval 290 nm-315 nm. An invisible light lamp is often referred to as “blacklight” (or often “black light”), also referred to as a UV-A light, Wood's lamp, or ultraviolet light, is a lamp that emits long-wave (UV-A) ultraviolet light and very little visible light, i.e., the output from the lamp appears black. Black light sources are essential when UV-A light without visible light is needed, particularly in observing fluorescence. The presently disclosed light emitting device may also be termed “Black Light”, because no visible light is emitted, but in contrast to normal black light devices, the presently disclosed light emitting device emits light around 290 nm-315 nm, which is part of the UVB range. This range turns out to be most efficient for stimulating the natural formation of ND3 in animals, such as piglets and mink kits. But the light emitted from the presently disclosed light emitting device may also comprise light in the UVA range from 315 to 380 nm, but that is not necessary-the most important is the light emitted from the device comprises light in the UVB range, in particular light from 290-305 nm, but no harmful UVC light below 280 nm and no visible light, such as from 380 nm- to 750 nm.

In an embodiment of the present disclosure the light emitting device is configured to reduce the microbial pressure in the animal nest. This may allow for significant reductions in the amount of antibiotics and other medicines whereby both the animal health is improved and cost-savings for the farmer are achieved. A further advantage realized by the present disclosure, in particular in relation to farms with pigs, piglets and minks, and mink kits is that methicillin resistant Staphylococcus aureus (MRSA) bacteria as well as other aerosol carried infections, such as virus infections, including coronaviruses, such as swine acute diarrhea syndrome coronavirus (SADS-CoV) can be removed significantly and thereby the health and well-being for both animals and farm workers is improved. A reduction in the microbial pressure can be achieved by disinfection with methods of the present disclosure.

Another aspect of the present disclosure relates to an animal nest for enhancing the natural formation of ND3 in animals in the nest, such as mink kits or piglets. In the preferred embodiment said pig nest comprises at least one of the presently disclosed light emitting devices, i.e. said at least one light emitting device is provided in the animal nest, such that light from the light emitting device is illuminating the inside of the animal nest. The light emitting device of this system would not disturb the sleep of the animals, such as piglets and mink kits, as it does not illuminate any visible light. Therefore, it can be used to illuminate inside the animal nests, in which the animals, such as piglets and mink kits, usually sleep, since the light does not disturb the sleeping animal. As an animal nest, and in particular a mink kit nest or a piglet nest, typically is a small cover less than 1 meter in height, in particular for piglets and suckling pigs, whereas a mink kit nest typically has a height of less than 20 cm, or even less than 10 cm, typically around 8 cm. This has the further advantage of bringing the light emitting device as close to the animals as possible, so that the light intensity reaching the animal is increased.

Yet another aspect of the present disclosure relates to a method for increasing the formation of ND3 in an animal by illuminating said animal with UVB light in a wavelength interval 290 nm-315 nm 8 hours a day, more preferably 16 hours a day, most preferably 24 hours a day. In the preferred embodiment the illumination should happen by using illuminating the presently disclosed light emitting device. By using the presently disclosed light emitting device which is not emitting any visible light or any harming UV light, but is emitting light in the wavelength interval 290 nm-315 nm it is possible to illuminate said animal only with light that enhances the formation of ND3 without the animal being exposed to any other wavelengths of light from the presently disclosed light emitting device. This further means that the light can be turned on at all times, day and night, without disturbing the night sleep of the animals by the presence of visible light. Hence the light emitting device of the present disclosure can be turned on without stressing the animal in its sleep or prevent it from falling asleep as visible light would otherwise do. Advantageously the light emitting device may hence be used during night-time as well as day-time in order to even further increase the formation of ND3 in the skin of the animal and/or decrease the microbial pressure of the animal nest.

In a further embodiment the presently disclosed light emitting device can also be used more generally for domesticated animals, i.e. in a stable or a pigsty where the animals spend the most of the day, in order to increase natural formation of ND3 in the animals. But then the UVB light source may be located too far away from the animals to have an effect on the animals' vitamin D production, and it is not all animals that even need to have the vitamin D production stimulated. Hence, the greatest effect is provided in animal nests where that UVB light source can be located close to the animals, such as piglets and mink kits which are in need to have their vitamin ND3 production stimulated in order to strengthen their immune system.

### DESCRIPTION OF THE DRAWINGS

The invention will in the following be described in greater detail with reference to the accompanying figures:

FIG. 1 shows one example of an animal nest (prior art)

FIG. 2 shows another example of an animal nest (prior art)

FIG. 3 shows yet another example of an animal nest (prior art)

FIG. 4 shows an example where piglets are relaxing in an animal nest (prior art)

FIG. 5 shows one example of a weaner nest (prior art)

FIG. 6 shows one example of a finisher nest (prior art)

FIG. 7 shows a spectrum of a light emitting device, according to an embodiment of the present disclosure

FIG. 8 shows a mink nest with mink kits, according to an embodiment of the present disclosure

FIG. 9 shows a mink nest with a light emitting device, according to an embodiment of the present disclosure

### DETAILED DESCRIPTION

It is a strong preference that the presently disclosed light emitting device, and the related methods, is configured such that no visible light is emitted. The term visible light, as used herein, refers to the portion of the electromagnetic spectrum that is visible to the naked eye. It is important to note that the visible spectrum differs for different species. Therefore, in a preferred embodiment of the present disclosure, visible light refers to light that can be detected by the naked eye of an animal, such as a pig, for example a piglet and/or suckling pigs, or a mink, for example a mink kit.

In a preferred embodiment of the present disclosure, visible light has a lowest wavelength of around 320 nm-380 nm. At the same time, in another preferred embodiment of the present disclosure, visible light has a highest wavelength of around 700 nm-750 nm. Wavelengths outside these ranges, i.e. below 320 nm-380 nm and above 700 nm-750 nm, do not belong to the category of visible light as used herein. Thereby visible light in specific embodiments of the present disclosure may be defined as light with a wavelength in the range between 320 nm-700 nm. In a further embodiment of the present disclosure, visible light is light with a wavelength in the range between 360 nm-700 nm. However, in a further embodiment of the present disclosure, visible light is defined as light with a wavelength in the range between 380 nm-700 nm. Visible light in further specific embodiments of the present disclosure is defined as light with a wavelength in the range between 320 nm-750 nm. In a further embodiment of the present disclosure, visible light is light with a wavelength in the range between 360 nm-750 nm. However, in a further embodiment of the present disclosure, visible light is defined as light with a wavelength in the range between 380 nm-750 nm.

The following definitions of NUV, MUV and FUV are used herein: NUV (Near Ultraviolet), 300-400 nm: Long-wave, black light, not absorbed by the ozone layer. MUV (Middle Ultraviolet): 200-300: Medium-wave, mostly absorbed by the ozone layer. FUV (Far Ultraviolet), 122-200 nm: Short-wave, germicidal, completely absorbed by the ozone layer and atmosphere. I.e. the term NUV/MUV it be understood to cover both wavelength ranges from 200 to 400 nm.

LED, as used herein, refers to a semiconductor light source that emits light when current flows through it, or a LED-chip, such as a surface-mounted-diode or a chip-on-board. By selection of different semiconductor materials, single-color LEDs can be made that emit light in a narrow band of wavelengths from near-infrared through the visible spectrum and into the ultraviolet range. As the wavelengths become shorter, because of the larger band gap of these semiconductors, the operating voltage of the LED increases. In general, an LED may refer to a light-emitting diode, while a LED-chip may refer to a chip comprising an LED. However, these two terms are used herein interchangeably. Manufacturing of LED-chips typically comprises MOCVD.

In an embodiment of the present disclosure, the light emitting device comprises at least one light source that is an LED and/or a LED-chip. The light emitting device may comprise at least one first monochromatic light source that is a LED and/or a LED-chip. In other embodiments, the light emitting device may comprise at least one first monochromatic light source that is a LED and/or a LED-chip, and at least one second monochromatic light source that is a LED and/or a LED-chip. In further embodiments the light emitting device may comprise at least one first monochromatic light source that is a LED and/or a LED-chip, at least one second monochromatic light source that is a LED and/or a LED-chip and at least one third monochromatic light source that is a LED and/or a LED-chip.

The piglet nest, also known as piglet creep, are sized according to regulations, and may be sized according to “Static space requirements for piglet creep area as influenced by radiant temperature”, E. F. Wheeler et al., 2008. American Society of Agricultural and Biological Engineers. In one embodiment of the present disclosure, the piglet nest area is less than 2 m², yet more preferably less than 1.5 m², even more preferably less than 1 m², yet even more preferably less than 0.75 m², most preferably less than 0.5 m². At the same time the height of the piglet nest is less than 2 m, preferably less than 1.5 m, more preferably less than 1 m, yet more preferably less than 0.75 m, even yet more preferably less than 0.5 m, most preferably less than preferably less than 0.4 m.

A mink nest, also referred to as a mink kit nest, is preferably provided as a sheltered environment, typically without or with only a small level of natural light. A mink nest in an animal farming facility may be provided as a cavity in hay, typically a closed lined (e.g. with hay and/or straw) cubicle with a single opening in one of the walls for entrance and exit. In an embodiment of the present disclosure, the light emitting device is configured to cover a mink kit nest, such as a cavity in hay.

Mink kits are born with a very immature immune system and with low serum concentrations of circulating immunoglobulins, similar to new-born piglets. Therefore, it is crucial that these animals, soon after birth, reach high blood concentrations of IgG through passive immunization by receiving IgG from the mother through colostrum (raw milk). In an embodiment of the present disclosure, the light emitting device is configured to emit one or more of the wavelengths 293 nm, 297 nm, and 301 nm. These wavelengths will stimulate the immune system of the mink kits, in the same way as the colostrum, thereby providing a significant boost to the immune system of the mink kits. In addition, the light emitting device may preferably be configured to minimize the microbial pressure in the mink nest, such as zoonotic diseases, including coronavirus.

One embodiment of the present disclosure relates to a light emitting device for use in an animal nest for enhancing the formation of ND3 in animals in the animal nest, comprising
at least one UVB light source, and

wherein the light emitting device is configured such that light in a wavelength interval of 290 nm-305 nm is emitted from the device, whereas UV light below 285 nm and visible light between 320 and 700 nm is not emitted from the device.

In an embodiment of the present disclosure, the light emitting device is configured for emitting monochromatic light having a wavelength in the interval of 290 nm-315 nm.

In a further embodiment of the present disclosure, the light emitting device comprises at least one first monochromatic light source configured for emitting light having a first wavelength, such as 293 nm, and at least one second monochromatic light source configured for emitting light having a second wavelength, such as 297 nm and a optionally at least one third monochromatic light source configured for emitting light having a third wavelength of 302 nm.

In a further and preferred embodiment of the present disclosure, the light emitting device comprises at least one first monochromatic light source configured for emitting light having a first wavelength between 290 nm-315 nm, such as 293 nm, preferably also at least one second monochromatic light source configured for emitting light having a second wavelength between 290 nm-315 nm, different from the first wavelength, such as 297 nm, and preferably also at least one third monochromatic light source configured for emitting light having a third wavelength between 290 nm-315 nm, different from the first and second wavelengths, such as 302 nm.

In that regard it is noted that light with a wavelength of around 293 nm may help to convert 7-dehydrocholesterol in the subcutaneous tissue in animals to vitamin ND3, which then enters the blood—and also the milk of a mink bitch.

It is further noted that light with a wavelength of around 297 nm may also help to convert 7-dehydrocholesterol in the subcutaneous tissue in animals to vitamin ND3, which then enters the blood—and also the milk female animal, such as mink bitch or a sow.

It is further noted that light with a wavelength of around 302 nm may help to convert the keratinocytes in the epidermis of animals to vitamin ND3, thereby strengthening the skin thereby reducing the risk of skin damage and skin cracks, thereby reducing the risk of MRSA infections.

In a further embodiment of the present disclosure, the light emitting device, such as the at least one light source, is configured for emitting polychromatic light, wherein at least part of the emitted polychromatic light have wavelength(s) in the interval of 290 nm-305 nm.

In a further embodiment of the present disclosure, the outer glass of the fluorescent light source is made of a material which is transparent for some or all light wavelengths in the interval 290 nm-305 nm and/or IR radiation.

In a further embodiment of the present disclosure, the outer glass of the fluorescent light source is made of a material absorbing light of at least some wavelengths outside the wavelength interval 290 nm-305 nm and/or IR radiation, for example a material absorbing visible light such as colored glass.

In a further embodiment of the present disclosure, the screen is transparent for some or all light wavelengths in the interval 290 nm-305 nm and/or IR radiation.

In a further embodiment of the present disclosure, the screen is absorbing at least some light wavelengths outside the wavelengths interval 290 nm-305 nm and/or IR radiation.

In a further embodiment of the present disclosure, the screen is configured to filter out or absorb all light emitted from the at least one light source which is not within the wavelength interval 290 nm-305 nm and/or IR radiation.

The present disclosure further relates to a method for increasing the formation of ND3 in an animal by illuminating said animal with UVB light 24 hours a day using a light emitting device which is not emitting UV light below 285 nm and not emitting visible light between 320 and 700 nm, but is emitting light in a wavelength interval 290 nm-305 nm.

In a further embodiment of the present disclosure, wherein, during day-time, said animal is exposed to light with wavelength(s) in the interval 290 nm-305 nm simultaneously with light of other wavelengths such as light from a halogen bulb or a linear fluorescent lamp.

In a further embodiment of the present disclosure, wherein, during night-time and/or day-time, the animal is only exposed to light with wavelength(s) in the interval 290 nm-305 nm.

In a further embodiment of the present disclosure, wherein, during night-time and/or day-time, the animal is only exposed to light with wavelength(s) in the interval 290 nm-305 nm and IR radiation.

In one embodiment of the present disclosure, at least one light source of the light emitting device is configured for emitting monochromatic and/or polychromatic light having a wavelength in the interval of 290 nm-315 nm. Alternatively, the light emitting device may be configured for emitting quasi-monochromatic light having its wavelength intensity peak within the interval of 290 nm-315 nm. As many light sources are not perfectly monochromatic the monochromatic light source or quasi-monochromatic light source may have some small degree of light emission which is not entirely monochromatic. Quasi-monochromatic light and/or polychromatic light may be considered as light radiation in which most of the energy is confined to a single wavelength or a very narrow waveband. An LED and/or a LED-chip is an example of a quasi-monochromatic light source. A laser is an example of a monochromatic light source. Hence, whenever referred to monochromatic light in the present disclosure the term monochromatic light should be considered light which is monochromatic, and/or quasi-monochromatic, and/or polychromatic, and hence a monochromatic light source should be considered a source which emits light in which most of the energy is confined to a single wavelength or a very narrow waveband. Hence monochromatic light sources of the present disclosure includes LEDs and/or a LED-chips. When referring to the wavelength of the light from a monochromatic light source the light wavelength may refer to the wavelength in which most of the energy of the light is confined or the peak position of the very narrow band of which most of the energy of the light is confined. The at least one light source of the light emitting device may be an LED and/or a LED-chip.

In an embodiment of the present disclosure the light emitting device comprises at least one first monochromatic and/or polychromatic light source configured for emitting light having a first wavelength, such as 293 nm, and at least one second monochromatic light source configured for emitting light having a second wavelength, such as 303 nm and/or 311 nm. It may be a preference that the second monochromatic light source is configured for emitting light having a wavelength of 311 nm. Thereby in an embodiment of the present disclosure, the first monochromatic light source is configured for emitting light in the interval 290 nm-305 nm and the second monochromatic light source is configured for emitting light having a wavelength of 311 nm. In a further embodiment of the present disclosure, the light source is configured to emit light in the interval 290-305 nm, having 311 nm, and light having wavelengths above 700 nm, more preferably above 750 nm, in principle within the interval of 700 nm-1 mm, more preferably within the interval of 750 nm-1 mm. It is a strong preference that the light source is configured to only emit said wavelengths, i.e. such that it does not emit other wavelengths.

The at least one light source of the presently disclosed light emitting device may be configured for emitting polychromatic light, wherein at least part of the emitted polychromatic light have wavelength(s) in the interval of 290 nm-320 nm, more preferably 290 nm-311 nm, most preferably 290 nm-305 nm. For example light composed of three different wavelengths centered around 293 nm, 297 nm, and 301 nm, respectively.

The light source may be a fluorescent light source. The fluorescent light source may be based on phosphor luminescence, such as from Sr₂LiSiO₄F, for example Ce³⁺, Mn²⁺ co-doped Sr₂LiSiO₄F. If the light source is a fluorescent light source the outer glass of the fluorescent light source may be made of a material which is transparent for some or all light wavelengths in the interval 290 nm-315 nm, such as 290 nm-305 nm. The outer glass of the fluorescent light source may also be made of a material absorbing light of at least some wavelengths outside the wavelength interval 290 nm-305, such as 290 nm-315 nm, for example a material absorbing visible light such as colored glass. This has the advantage that all light emitted by the fluorescence process, which is not within the wavelength interval 290 nm-315 nm, such as 290 nm-305 nm, can be absorbed by the outer glass of the fluorescence light source and hence result in the light emitting device only emitting light in the wavelength interval 290 nm-315 nm, such as 290 nm-305 nm. The fluorescent light source may be a compact fluorescent lamp or a linear fluorescent lamp.

Also, other light sources used in the present disclosure may use a similar approach in which the material defining the circumference of the light source is made from a material which absorbs light outside the interval of 290 nm-305 nm, such as outside the interval of 290 nm-315 nm. The material defining the circumference may hence serve more than one purpose as it may also function as protection of the light source and/or securing suitable conditions, such as a suitable atmosphere, for the light source to work. Examples may be colored glass used for a light bulb, or colored glass used for a fluorescence light source.

In one embodiment the presently disclosed light emitting device comprising at least one screen covering the at least one light source. This may for instance be relevant if the light source needs to be protected from the environment of which it is used. Preferably the screen is transparent for some or all light wavelengths in the interval 290 nm-315 nm, such as some or all light wavelengths in the interval 290 nm-305 nm, so that is does not prevent the light in this interval from leaving the light emitting device nor decreases the intensity of the light wavelengths in the interval 290 nm-315 nm, such as in the interval 290 nm-305 nm.

In one embodiment of the present disclosure the light emitting device may comprise a screen and one or more light source(s) in the form of LED(s) and/or a LED-chip(s). The light of the LED and/or a LED-chip is monochromatic in the sense of the definition of this present disclosure and hence there is a fraction of the light from LED(s) and/or a LED-chips which does not have the dominant wavelength of the LED and/or a LED-chip. Hence an LED and/or an LED-chip emitting monochromatic light in the wavelength interval 290 nm-315 nm, such as in the interval 290 nm-305 nm may have some fraction of light outside of this interval. Here it is important that these wavelengths are absorbed within the light emitting device in order for the device to only emit light within the wavelength interval 290 nm-315 nm, such as only emit light within the wavelength interval 290 nm-305 nm. The light emitted from the light source which is not within the wavelength interval 290 nm-315 nm and/or within the interval 290 nm-305 nm should hence be absorbed within the light emitting device, for example by the screen. Hence the screen may be made of a material absorbing all wavelengths not within the wavelength interval 290 nm-305 nm or the interval 290 nm-315 nm emitted by the light source(s), but be transparent to all wavelengths within the wavelength interval 290 nm-305, or all wavelengths within the wavelength interval 290 nm-315 nm, emitted by the light source(s). In this example the light source has been discussed as being an LED and/or an LED-chip, but the light source may also be a fluorescence light source or any other light source emitting some light within the wavelength interval 290 nm-315 nm and some light outside the wavelength interval 290 nm-315 nm.

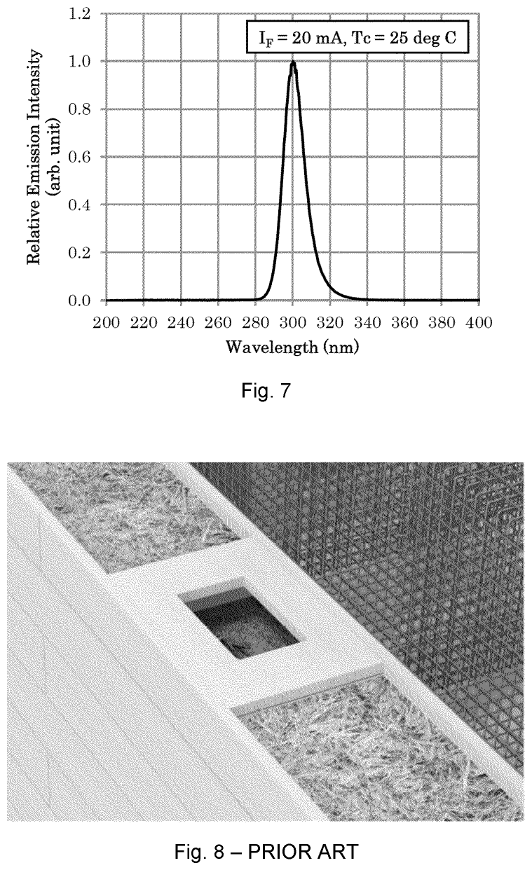

An example of a light spectrum from an UV LED with a center wavelength of around 300 nm is shown in FIG. 7. As seen from the spectrum of said figure, an LED and/or an LED-chip is not necessarily perfectly monochromatic and tails of the spectrum can lead to a certain emittance of light in other spectral regions, such as the FUV spectrum (below 200 nm), or in the visible spectrum. A screen covering the at least one light source(s) of the presently disclosed light emitting device may be configured to absorb the unwanted light wavelengths, e.g. outside the MUV (200-300 nm) and NUV (300-400 nm) wavelength intervals, or even outside the preferred 290 nm-315 nm wavelength interval.

The screen may be absorbing at least some light wavelengths outside the wavelengths interval 290 nm-305 nm, such as outside the wavelength interval 290 nm-315 nm. For example, may the screen absorb light with wavelengths in the visible spectrum (such as 380 nm-750 nm). Hence the screen may be made from a material absorbing at least some light having wavelengths within the spectrum of visible light, such as colored glass. The screen may hence be configured to filter out or absorb all light emitted from the at least one light source which is not within the wavelength interval 290 nm-315 nm, such as within the wavelength interval 290 nm-305 nm. This will serve to ensure that all light leaving the light emitting device is within the wavelength interval 290 nm-315 nm, such as within the wavelength interval 290 nm-305 nm, even if not all the light emitted from the light source(s) is/are within the interval. The screen may have the shape of a bulb, a tube or a disc. As the screen may cover the light source(s) the screen may define the overall shape of the light emitting device. In case of a fluorescent light source the screen may have the dual function of providing the fluorescent luminescence but also absorbing the unwanted wavelengths such that not visible light and no FUV light is emitted from the light emitting device.

Infrared light (IR) is electromagnetic radiation with wavelengths longer than those of visible light, i.e. generally invisible to the human eye, although IR at wavelengths up to 1050 nm can be seen by humans under certain conditions. IR wavelengths extend from the nominal red edge of the visible spectrum at 700 nm all the way up to 1 mm. Most of the thermal radiation emitted by objects near room temperature is infrared. IR carries radiant energy and can therefore be used to transfer heat, which is utilized in an infrared heater, aka an infrared heater.

Infrared heaters can be classified by the wavelength bands of infrared emission:

Near-infrared for the range from 780 nm to 1400 nm (0.78-1.4 μm), these emitters are also named bright because still some visible light is emitted;

Short wavelength infrared for the range of 1.4-3 μm;

Medium infrared, or mid wavelength infrared, for the range of 3-8 μm;

Long wavelength infrared for the range of 8-15 μm;

Far infrared for everything above 15 μm, typically up to around 1000 μm, i.e. 1 mm.

These definitions of the infrared ranges are also used herein. Near-infrared and short wavelength infrared are also referred to as “reflected infrared”, whereas Medium infrared and Long wavelength infrared may be referred to as “thermal infrared”.

In one embodiment of the present disclosure light emitting device is emitting infrared radiation, such that the light emitting device is emitting light having wavelengths within the interval of 290 nm-315 nm, such as within the interval of 290 nm-305 nm, and light having wavelengths above 700 nm, more preferably above 750 nm, in principle within the interval of 700 nm-1 mm, more preferably within the interval of 750 nm-1 mm. An essential feature is still that no visible nor damaging light is emitted from the light emitting device. Hence in this case of the light emitting device also emitting infrared light, the light emitting device may serve a dual purpose as it would both warm the animal and enhance the formation of vitamin D3 in its skin. Furthermore, it may be possible that the light emitting device could comprise one or more light source(s) for emitting the infrared light and not emitting light within the interval of 290 nm-315 nm, such as the interval of 290 nm-305 nm. This/these infrared light source(s) may also produce some visible light. In this case the visible light should be absorbed by other elements of the light emitting device, for example by a screen as discussed previously, so that no visible light would be emitted from the light emitting device.

The light emitting device may be used within the field of enhancing the natural formation of ND3 in the skin of living animals. Preferably the light emitting device is turned on for long periods of time, possibly it will be turned on constantly during its entire lifetime. Hence it is preferable that the light emitting device is running at a low power. In the preferred embodiment the power of the light emitting device is in the interval of 5 W-300 W, such as 5 W-20 W or 5 W-50 W or 50 W-100 W or 100 W-200 W or 200 W-300 W. It is preferred that the light emitting device of the present disclosure is energy- and cost-effective.

In a preferred embodiment of the present disclosure, the light emitting device is configured such that only a single lamp housing and/or lamp socket is required for accommodating said device. The light emitting device may thereby be a polychromatic lamp, configured to emit light with wavelengths and intensities as disclosed herein. A single polychromatic lamp offers significant advantages with respect to a combination of multiple lamps, wherein each provides only a part of the spectrum of the light emitting device, for producing a similar light. For example, a single polychromatic light emitting device allows for substantially reduced requirements of the electrical installations, including the requirements on the switching cabinet and the requirements on the number of lamp sockets for accommodating the lamp. Further, a single polychromatic lamp or a composite LED lamp can offer a more even illumination than what several lamps, each providing only a part of the spectrum of the light emitting unit, can offer. Thereby, the presently disclosed light emitting device may advantageously allow for formation of ND3 in the skin of living animals, decrease the bacterial pressure in the animal nest, while decreasing the overall costs and space requirements.

Another aspect of the present disclosure relates to an animal nest, such as a mink nest or a piglet nest, for enhancing the natural formation of ND3 in animals in the nest comprising at least one of the presently disclosed light emitting device which may be installed in the animal nest, such that light from the light emitting device(s) is/are illuminated into the inside of the animal nest.

Too much UV light is harmful to many living beings, and in particular short wavelength NUV/MUV light can be harmful with too much exposure. It does therefore seem counterintuitive to place a UV light source, such as a NUV/MUV light source, in an animal nest, such as a piglet nest or a mink nest. It may be considered a potentially harmful UV device which is located very close to very young animals. But with careful considerations of the light emittance and exposed area, burning of the animals by too much UV exposure can be prevented and this can in particular be ensured by adjusting the wattage of the UV light emitting device but also the distance between the UV light source and the animals. The maximal allowed daily UV exposure for an animal is 50.000 mJ/mm². As an example a 10 watt fluorescent lamp emits approx. 2 Watt UV light. The presently disclosed UV light emitting device can be configured for illumination 24/7, but the piglets only stay inside the piglet creep for less than one hour at a time because they must breastfeed for every 45 minutes. The illuminated area inside a piglet nest is typically ø 50 cm=1.963 mm². I.e. inside the piglet creep the piglets are exposed to 2 W×1 hour/1.963 mm2=3668 mJ/mm² per hour, i.e. in this example they can be inside the piglet creep for more than 13 hours in succession without getting burned.

Hence, the presently disclosed UV light emitting device is in one embodiment configured such that the radiant flux emitted from the UV light emitting device is less than 10 W, more preferably less than 5 W, most preferably less than 2 W, possibly even below 1 W. This kind of emitted flux would mostly be relevant for use in animal nests, and in particular piglet nests and mink nests. For porker unit nests the necessary radiant flux might be larger because the height of the nest is larger. Hence, the presently disclosed UV light emitting device is in another embodiment configured such that the radiant flux emitted from the UV light emitting device is less than 30 W, more preferably less than 20 W, most preferably less than 15 W.

Even a screen or an outer glass may become too hot and the presently disclosed UV light emitting device and the light emitting device, such as a IR heat device, may be provided with a protective grate to protect the animals from the possibly hot screen, outer glass and/or light source.

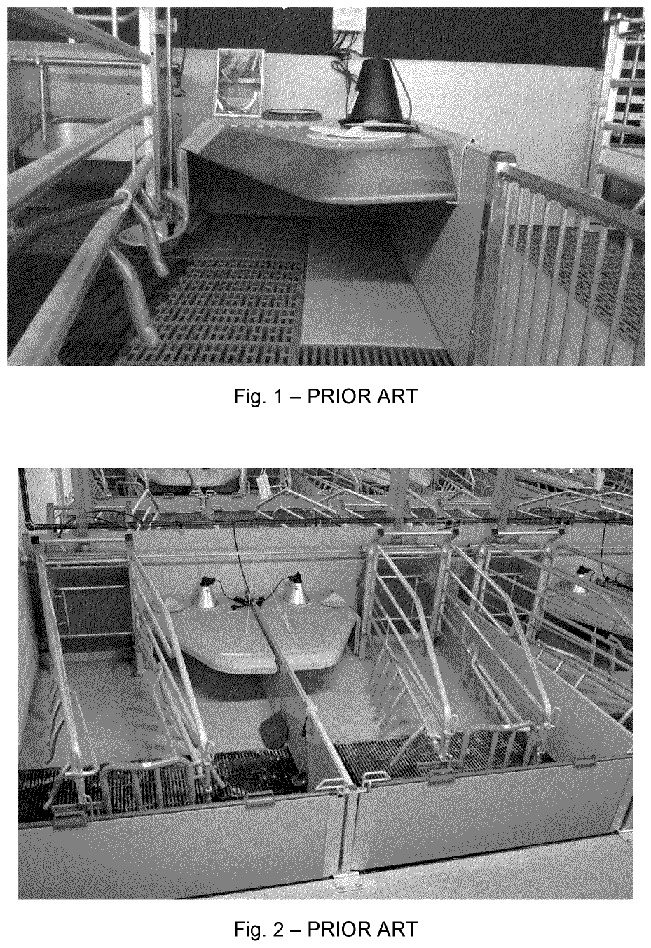

A piglet nest may be considered a designated area for the new-born and young piglets to relax, see for example FIGS. 1-4 showing prior art piglet nests. Piglets need to relax in darkness and hence the piglet nests normally have a non-transparent roof to make the area darker. Typically, piglet nests are equipped with a heating lamp to also keep the piglets warm while they relax in their nest. A nest may have 2-3 side walls, leaving an area open for the animal to enter, but otherwise shielding the animals as much as possible. The same conditions typically applied to minks, such as mink kits, wherein the minks need to relax in darkness and hence the mink nests may have a non-transparent roof to make the area darker.

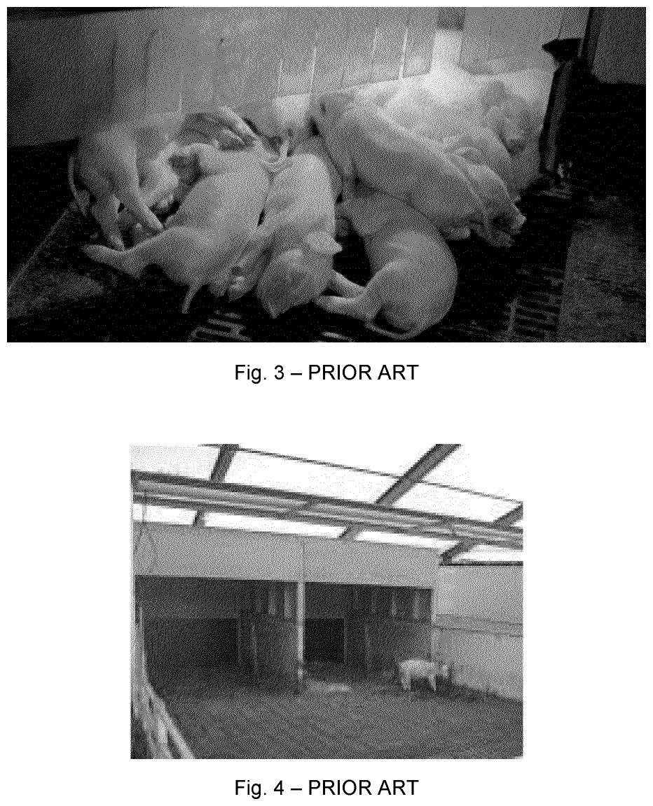

Heating lamps can be seen in FIGS. 1 and 2 where the lamps are mounted on top of the roof of the piglet nests and shine light through a window or hole in the roof such that the inside of the nest is illuminated and heated by the heating lamp. Normal prior art heat lamps for piglet nests and weaner nests are typically 150 watt infrared heating lamps emitting a considerable amount of visible light, as also seen in FIG. 3. The animals in the creep are certainly kept warm but they are also disturbed by the visible light and the high wattage of the lamps results in a lifetime of only around 3000 hours.

A piglet nest may be provided with 2-3 side walls leaving an area open for the animal to enter, but otherwise shielding the animals as much as possible. Sometimes a pig nest is merely a horizontal shield mounted in a corner of the pigsty in a height of 20-100 cm from the floor, such that the sides of the corner form the side walls and the horizontal shield forms the roof of the pig creep.

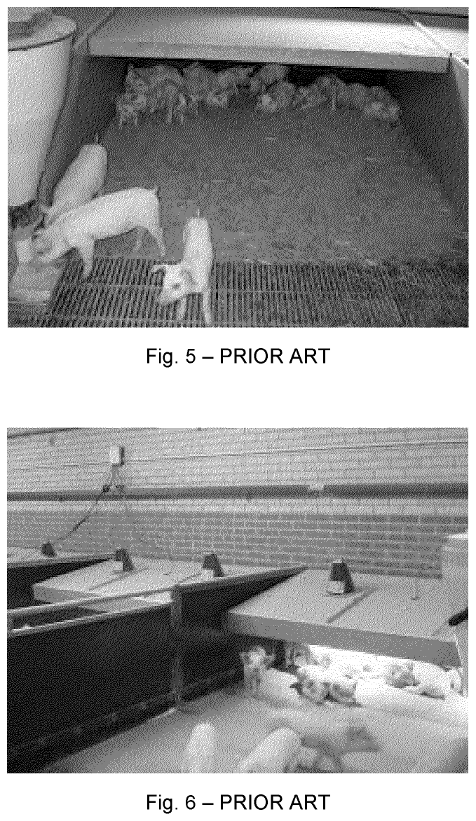

A finisher nest is typically a bit higher, from 1 to 2 meters in height typically, as also shown in FIGS. 4-6 with examples of prior art finisher creeps. 4 shows two finisher creeps side by side in one end of pigsty and finisher in the pigsty can take cover in each of the finisher creeps. The distance from the floor to the roof of the creeps are about 1.5 to 2 meters. In order for an NUV/MUV light source installed in the roof to have any effect it would have to be quite powerful. The finisher nest in FIG. 5 has a lower height, approx. 1 meter, and power requirements for an NUV/MUV light source as well as an IR heat source would be somewhat lower compared to FIG. 4. A number of pigsties are located next to each other in FIG. 6, each pigsty is provided with a finisher nest, and each finisher nest is provided with two heating lamps mounted in the roof to heat the inside of the finisher nests. The finisher nests in FIG. 6 have a comparable height to the one in FIG. 5, but as seen in FIG. 6 the roofs are slanted towards the pigsty and are provided with an edge to partly screen the heat and light emitted from the heating lamps. As seen in FIG. 6 the heating lamps provide a clear glow of visible light, i.e. the pigs might be heated but will have trouble sleeping deeply if the heating lamps are turned on all the time. The finisher nests in FIGS. 4 and 5 are not provided with heating lamps, because finishers do not necessarily need additional heat. However, the shielding provided by the finisher nests can be comfortable for the finisher and they often take cover in the nests, even without heating lamps. Hence, the presently disclosed UV light emitting device can advantageously be provided in finisher nests such that the finisher can be illuminated with NUV/MUV light to stimulate the formation of ND3, but without disturbing the animals.

As also stated above the present disclosure further relates to an IR heat emitting device for use in an animal nest, such as a pig nest, for stimulating the growth of piglets and weaners in the nest, comprising at least one mid-IR source for emitting mid-IR radiation in a wavelength interval of approx. 3-15 μm for heating animals in the animal nest, and the IR heat emitting device is configured such that visible light is not emitted from the device. In that way the animals in the nest can be kept warm without disturbing their sleep with visible light, i.e. the animals can enter the deep sleep stage during their sleep cycles such that stimulation of growth is enhanced leading to improved mental health due to better sleep, faster growth, bigger animals, stronger bones, etc. i.e. generally improved well-being of the animals. A mid-infrared source is also more energy efficient such that the wattage can be reduced to approx. 50 watt with an increase in the lifetime of the presently disclosed mid-IR source to about 50.000 hours.

In the preferred embodiments heat emission of the infrared source is based on at least one carbon fiber element, e.g. a carbon filament or based on a ceramic heating element e.g. a ceramic filament. The configuration of the carbon fiber element and/or ceramic filament, and the power control thereof, determines the light spectrum emitted from the IR source. Hence, a skilled person will know how to configure and control an infrared source based on carbon fiber element and/or ceramic filament, such that mid-IR radiation is emitted therefrom, in particular such that no visible light is emitted therefrom, preferably such that only light above 1000 nm, or even above 1500 nm, most preferably above 2000 nm, possibly even above 2500 nm is emitted therefrom.

The IR heat emitting device preferably comprises at least one screen covering the at least one light source. The screen can help with reducing or eliminating emittance of visible light from the IR source, e.g. if the screen is configured for absorbing light wavelengths below 1000 nm, more preferably in the visible range of 360 to 700 nm. The screen is preferably transparent for some or all light wavelengths above 1000 nm. The screen can for example be made from a material absorbing at least some light having wavelengths within the spectrum of visible light, such as colored glass, such as black glass. The screen may have the shape of a bulb, a tube or a disc, etc.

An mid-infrared heat source with not visible light is much more efficient than a standard heat lamp and the power consumption of such a lamp can be reduced significantly compared to a standard heat lamp, which will also increase the lifetime of the light source of the presently disclosed IR heat emitting device. As no visible light is emitted the presently disclosed IR heat emitting device can be turned on 24 hours a day. Temperature control inside the animal nest, such as a piglet nest, can be provided by a temperature sensor inside the nest and a feedback loop to a control unit of the IR heat emitting device. However, as the source of heat is mid-IR it can be more suitable to monitor the temperature of skin of one or more animals inside the nest instead of monitoring the air temperature inside the nest.

In one embodiment the at least one light emitting device(s) is/are situated in the roof of the nest and/or in a in the wall(s) of the nest and/or in the floor of the nest. By installing the light emitting device in the circumference of the animal nest the light emitting device can be brought very close to the animals as they lie inside their nest. Furthermore, the walls or roof of the nest does not screen for the invisible light emitted by the light emitting device. Preferably the at least one light emitting device is installed such that the distance from the light emitting device to the floor of the animal nest is less than 60 cm, more preferably less than 50 cm, more preferably less than 40 cm, more preferably less than 30 cm, more preferably less than 20 cm and most preferably less than 10 or even 8 cm. Such low nests of less than 10 cm are typical for mink nests. The at least one light emitting device may be fastened to the nest by being partly inserted into a hole in the nest roof or by being partly inserted into a hole in the side walls. For example, the hole may reflect the shape and size of the light emitting device, or at least the part of the light emitting device which emits light.

In the preferred embodiment the animal nest, such as a piglet nest, is further comprising at least one source of heat, such as an infrared heating lamp. For instance, the heat source could be an infrared lamp, an infrared carbon film, an infrared panel or an infrared pad. In a further embodiment the animal nest is further comprising at least one infrared light source not emitting any visible light. For instance, the infrared source could be an infrared lamp, an infrared carbon film, an infrared panel or an infrared pad, which should not emit any visible light. In this manner the animal lying inside the nest receives light in the wavelength interval 290 nm-315 nm, such as in the wavelength interval 290 nm-305 nm, in order to secure the enhanced formation of ND3, along with infrared radiation in order to keep it warm, but without any visible light being illuminated into the animal nest. Hence the animal can be kept warm and healthy without stressing the animal and without disturbing the sleep of the animal.

Yet another aspect of the present disclosure is a method for increasing the formation of ND3 in an animal by illuminating said animal with UVB light 24 hours a day using a light emitting device which is not emitting UV light below 285 nm and not emitting visible light, such as between 380 nm to 750 nm, but is emitting light in a wavelength interval 290 nm-315 nm, such as in the wavelength interval 290 nm-305 nm.

Preferably the light emitting device of the presently disclosed method is the presently disclosed light emitting device. More preferably, in the presently disclosed method, the animal is illuminated using the presently disclosed animal nest.

In the preferred embodiment of the presently disclosed method the light source is situated no longer than 1 meter away from the animal, more preferable no longer than 75 cm away from the animal, more preferably no longer than 50 cm away from the animal, more preferable no longer than 40 cm away from the animal, more preferable no longer than 30 cm away from the animal, more preferable no longer than 20 cm away from the animal, most preferable no longer than 10 or even 8 cm away from the animal, especially in case of mink kits.

During the day-time the animal may be exposed to light with wavelength(s) in the interval 290 nm-315 nm, such as in the wavelength interval 290 nm-305 nm simultaneously with light of other wavelengths such as light from a halogen bulb or a linear fluorescent lamp. For instance, this may refer to a pigsty- or stable-like environment in which the light inside the stable is turned on while also the light emitting device illuminating the animals with light in the wavelength interval 290 nm-315 nm, such as in the wavelength interval 290 nm-305 nm is on.

Preferably during night-time and day-time, said animal is only exposed to light with wavelength(s) in the interval 290 nm-315 nm, such as in the wavelength interval 290 nm-305 nm. During the night it is normal procedure to turn off the normal light of the stable and let the animal sleep or relax in the dark. However, in the presently disclosed method there is no need to turn off the light emitting device as the light does not disturb the sleep of the animal, however the light still helps the animal increase its natural formation of ND3. Keeping the light emitting device on all the time means that the animal can form ND3 day and night.

More preferably, during night-time and day-time, the animal is only exposed to light with wavelength(s) in the interval 290 nm-315 nm, such as in the wavelength interval 290 nm-305 nm and to IR radiation. In this manner the animal can form ND3 and be kept warm during night and day, without being disturbed by visible light.

The use of the presently disclosed light emitting device for example in the presently disclosed animal nest and/or using the presently disclosed method can be used to increase the natural formation of vitamin D3 in the animal. By using a light source which only emits light within the wavelength interval 290 nm-315 nm, such as in the wavelength interval 290 nm-305 nm it is possible to illuminate an animal using only this invisible light without disturbing the sleep or relaxation of the animal with visible light.

Illumination of an animal, such as a piglet or a mink kit, will result in a more effective formation of ND3 in the skin of the animal. This will result in healthier animals and decreasing mortality within a population of animals. Another desirable effect of these health improvements is that it enables the breeder to reduce the use of antibiotics which will be an important step to avoid development of multi-resistant bacteria. Another positive effect of an enhanced formation of ND3 is a better absorption of the phosphorus and calcium in the animal feed which will have a positive environmental effect for the production of feed. Farm animals with a high content of ND3 in their body produce milk and meat with a higher level of ND3 content and serve as an improvement of the humans' diet-based vitamin D3 absorption.

The term “natural light” used in this disclosure refers to the sunlight as it arrives at the face of the earth with the normal distribution of intensity in the spectrum.

UVB light may be defined as the range of wavelengths in the spectrum 280 nm-315 nm. Especially the light in the interval 290-305 nm has proven most effective for production of ND3 whereas light having wavelengths below 285 nm is harming for animals like piglet and mink kits. Preferably the presently disclosed light emitting device is exclusively illuminating with the light of the wavelengths which are most effectively generating ND3 in the skin of the animal in question. In an alternative embodiment of the present disclosure the light emitting device is exclusively illuminating with the light of the wavelengths which are most effectively generating ND3 in the skin of the animal in question in addition to light with a wavelength of, or of around, 290-305 nm.

The strategy of using colored glass in the outer surface of a light source is for example sometimes used in the field of black lights wherein only a small amount of visible light should be emitted by the light source. The present invention may be considered a type of black light but without any emission of visible light.

The light emitting device of the present disclosure not emitting any visible light may be interpreted as the light emitting device not emitting any visible amount of visible light.

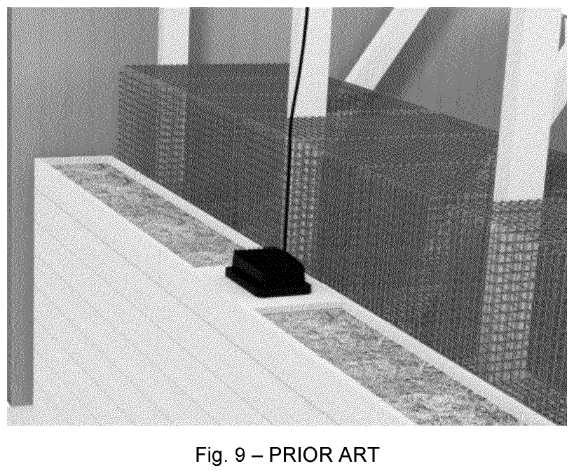

FIGS. 8-9 shows a mink nest, according to a specific embodiments of the present disclosure. The nest is provided in a cavity formed of hay. In FIG. 9, it can be seen how the light emitting device is arranged to cover the top of the mink nest, thereby irradiating the minks within the nest. The light emitting device may for example advantageously be configured to emit one or more of the wavelengths 293 nm, 297 nm, and 302 nm. These wavelengths will stimulate the immune system of the mink kits, in the same way as colostrum from the mink mother. Thereby providing a significant boost to the immune system of the mink kits. In addition, the light emitting device may preferably be configured to minimize the microbial pressure in the mink nest, such as zoonotic diseases, including coronavirus.

Items
1. An UV light emitting device for use in a pig creep for enhancing the formation of ND3 in animals in the pig creep, comprising
   at least one NUV/MUV light source, and
   wherein the UV light emitting device is configured such that light in a wavelength interval of 290 nm-305 nm is emitted from the UV light emitting device, whereas UV light below 285 nm and visible light between 360 and 1000 nm is not emitted from the UV light emitting device.
2. The UV light emitting device according to item 1 wherein the at least one light source is configured for emitting monochromatic light having a wavelength in the interval of 290 nm-305 nm.
3. The UV light emitting device according to any preceding items comprising at least one first monochromatic light source configured for emitting UV light having a first wavelength, such as between 292 and 294 nm, such as 293 nm.
4. The light emitting device according to any preceding items comprising at least one first monochromatic light source configured for emitting light having a first wavelength between 290 nm-315 nm, such as 293 nm, and
   at least one second monochromatic light source configured for emitting light having a second wavelength between 290 nm-315 nm, different from the first wavelength, such as 297 nm, and
   at least one third monochromatic light source configured for emitting light having a third wavelength between 290 nm-315 nm, different from the first and second wavelengths, such as 302 nm.
5. The UV light emitting device according to any preceding items comprising at least one second monochromatic light source configured for emitting UV light having a second wavelength, such as between 296 and 298 nm, such as 297 nm.
6. The UV light emitting device according to any preceding items comprising at least one third monochromatic light source configured for emitting UV light having a third wavelength, such as between 301 and 303 nm, such 302 nm.
7. The light emitting device according to any preceding items comprising at least one first monochromatic light source configured for emitting light having a first wavelength, such as 293 nm, and
   at least one second monochromatic light source configured for emitting light having a second wavelength, such as 303 nm and/or 311 nm.
8. The UV light emitting device according to any preceding items, wherein the at least one light source is an LED and/or an LED-chip.
9. The UV light emitting device according to any preceding items wherein the at least one light source is configured for emitting polychromatic light, wherein at least part of the emitted polychromatic light have wavelength(s) in the interval of 290 nm-305 nm.
10. The UV light emitting device according to any of the preceding items, wherein the at least one light source is a fluorescent light source.
11. The UV light emitting device according to any of the preceding items, wherein the light source is a fluorescent light source based on phosphor luminescence, such as from Sr₂LiSiO₄F, for example Ce³⁺, Mn²⁺ co-doped Sr₂LiSiO₄F
12. The UV light emitting device according to any preceding items 9-11, comprising outer glass for covering at least part of the fluorescent light source and wherein the outer glass is made of a material which is transparent for some or all light wavelengths in the interval 290 nm-305 nm.
13. The UV light emitting device according to any preceding items 9-12, wherein the outer glass of the fluorescent light source is made of a material absorbing light of at least some wavelengths outside the wavelength interval 290 nm-305 nm, for example a material absorbing visible light such as coloured glass, such as black glass.
14. The UV light emitting device according to any preceding items, wherein the fluorescent light source is a compact fluorescent lamp or a linear fluorescent lamp.
15. The UV light emitting device according to any preceding items, comprising at least one screen covering the at least one light source.
16. The UV light emitting device according to items 14, wherein the screen is transparent for some or all light wavelengths in the interval 290 nm-305 nm.
17. The UV light emitting device according to items 14-15, wherein the screen is absorbing at least some light wavelengths outside the wavelengths interval 290 nm-305 nm.
18. The UV light emitting device according to items 14-16, wherein the screen is made from a material absorbing at least some light having wavelengths within the spectrum of visible light, such as coloured glass, such as black glass.
19. The UV light emitting device according to items 14-17, wherein the screen is configured to filter out or absorb all light emitted from the at least one light source which is not within the wavelength interval 290 nm-305 nm.
20. The UV light emitting device according to any of the items 14-18, wherein the screen has the shape of a bulb, a tube or a disc.
21. The UV light emitting device according to any preceding items, configured such that the radiant flux emitted from the UV light emitting device is less than 300 W more preferably 10 W, more preferably less than 5 W, most preferably less than 2 W.
22. The UV light emitting device according to any preceding items, wherein the power of the UV light emitting device is in the interval of 2 W-300 W, such as 2 W-20 W or 2 W-50 W or 50 W-100 W or 100 W-200 W or 200 W-300 W.
23. An infrared (IR) heat emitting device for use in a pig creep for heating animals in the creep, comprising
   at least one thermal infrared source, and
   wherein the IR heat emitting device is configured such that radiation in a wavelength interval of between 3 μm and 15 μm, preferably between 8 μm and 15 μm, is emitted from the IR device, whereas radiation below 3 μm, most preferably below 8 μm, is not emitted from the IR device and wherein no visible light is emitted from the IR device.
24. The IR heat emitting device according to item 23, wherein emittance of the IR source is based on at least one carbon fiber element and/or at least one ceramic element.
25. The IR heat emitting device according to any preceding items 23-24, comprising at least one screen covering the at least one IR source.
26. The IR heat emitting device according to item 25, wherein the screen is transparent for some or all light wavelengths above 1000 nm, preferably above 3000 nm.
27. The IR heat emitting device according to items 25-26, wherein the screen is absorbing light wavelengths below 1000 nm, more preferably in the visible range of 380* to 700 nm.
28. The IR heat emitting device according to items 25-27, wherein the screen is made from a material absorbing at least some light having wavelengths within the spectrum of visible light, such as coloured glass, such as black glass.
29. The IR heat emitting device according to any of the items 25-28, wherein the screen has the shape of a bulb, a tube or a disc.
30. A radiation system for a pig creep comprising at least one of the UV light emitting devices according to any of items 1-22 and/or at least one of the IR heat emitting devices of any of items 23-29.
31. A pig creep for enhancing the natural formation of ND3 in animals in the creep, comprising at least one UV light emitting device according to any of the items 1-22, wherein the at least one light emitting device is installed in the pig creep, such that light from the light emitting device is illuminating the inside of the pig creep.
32. The pig creep according to item 31, wherein the at least one light emitting device is situated in the roof of the creep and/or in a in the wall(s) of the creep and/or in the floor of the creep.
33. The pig creep according to items 31-32, wherein the at least one light emitting device is fastened to the creep by being partly inserted into a hole in the creep roof or side walls.
34. The pig creep according to item 31-33, further comprising at least one source of heat, such as an infrared heating lamp.
35. The pig creep according to item 31-34, further comprising at least one of the IR heat emitting devices of any of items 23-29.
36. The pig creep according to items 31-35, wherein at least one light emitting device is installed such that the distance from the light emitting device to the floor of the pig creep is less than 200 cm, more preferably less than 150 cm, even more preferably less than 100 cm, yet more preferably less than 50 cm, most preferably less than 30 cm.
37. The pig creep according to items 31-36, wherein the pig creep is a piglet creep, such as a piglet creep for suckling pigs and/or a piglet creep for weaners, preferably with a height of the creep of less than 1 meter.
38. The pig creep according to items 31-36, wherein the pig creep is a finisher creep for finishers, preferably with a height of the creep of less than 2 meters.
39. A method for increasing the formation of ND3 in an animal by illuminating said animal with NUV/MUV light 24 hours a day using a light emitting device which is not emitting UV light below 285 nm and not emitting visible light between 320 and 700 nm, but is emitting light in a wavelength interval 290 nm-305 nm.
40. The method according to item 37 wherein said light emitting device is the light emitting device of items 1-22.
41. The method according to item 37 wherein said animal is illuminated using the pig creep of items 31-38.
42. The method of items 37-39 wherein the light source is situated less than 2 meter from and/or above the animal, more preferably less than 1.5 meter from and/or above the animals, even more preferable less than 1 meter from and/or above the animal, yet more preferably less than 50 cm from and/or above the animal, most preferable less than 30 cm from and/or above the animal.
43. The method according to items 37-40 wherein, during day-time, said animal is exposed to light with wavelength(s) in the interval 290 nm-305 nm simultaneously with light of other wavelengths such as light from a halogen bulb or a linear fluorescent lamp.
44. The method according to items 21-41 wherein, during night-time, said animal is only exposed to light with wavelength(s) in the interval 290 nm-305 nm.
45. The method according to items 37-41 wherein, during night-time, said animal is only exposed to light with wavelength(s) in the interval 290 nm-305 nm and IR radiation.

1. A light emitting device for use in an animal nest, comprising:
at least one light source, and
wherein the light emitting device is configured such that light in a wavelength interval of 290 nm-315 nm is emitted from the device, whereas UV light below 285 nm and visible light between 380 and 750 nm are not emitted from the device.

2. (canceled)

3. (canceled)

4. The light emitting device according to claim 1, wherein the at least one light source is configured for emitting monochromatic light having a wavelength in the interval of 290 nm-315 nm.

5. The light emitting device according to claim 1, further comprising at least one first monochromatic light source configured for emitting light having a first wavelength between 290 nm-315 nm, and
at least one second monochromatic light source configured for emitting light having a second wavelength between 290 nm-315 nm and different from the first wavelength, and
at least one third monochromatic light source configured for emitting light having a third wavelength between 290 nm-315 nm and different from the first and second wavelengths.

6. The light emitting device according to claim 1, further comprising at least one first monochromatic light source configured for emitting light having a first wavelength of 293 nm, and
at least one second monochromatic light source configured for emitting light having a second wavelength of 297 nm, and
at least one third monochromatic light source configured for emitting light having a third wavelength of 302 nm.

7. (canceled)

8. The light emitting device according to claim 1, wherein the at least one light source is configured for emitting polychromatic light, wherein the emitted polychromatic light includes a wavelength in the interval of 290 nm-315 nm.

9. The light emitting device according to claim 1, wherein the at least one light source is a fluorescent light source.

10. (canceled)

11. The light emitting device according to claim 9, wherein the fluorescent light source comprises an outer glass transparent to at least some wavelengths in the interval 290 nm-315 nm and/or to IR radiation.

12. The light emitting device according to claim 9, wherein the outer glass of the fluorescent light source comprises a material absorbing light of at least some wavelengths outside the wavelength interval 290 nm-315 nm and/or IR radiation.

13. (canceled)

14. The light emitting device according to claim 1, further comprising at least one screen covering the at least one light source, the screen optionally being transparent to at least some light wavelengths in the interval 290 nm-315 nm and/or to IR radiation.

15. (canceled)

16. The light emitting device according to claim 14, wherein the screen absorbs at least some light wavelengths outside the wavelengths interval 290 nm-315 nm and/or IR radiation.

17. The light emitting device according to claim 14, wherein the screen comprises a material absorbing at least some light having wavelengths within the spectrum of visible light.

18. The light emitting device according to claim 14, wherein the screen is configured to filter out or absorb all light emitted from the at least one light source that is not within the wavelength interval 290 nm-315 nm and/or is IR radiation.

19. The light emitting device according to claim 1, further comprising an infrared (IR) heat emitting device comprising at least one thermal infrared source, and wherein the IR heat emitting device is configured such that radiation in a wavelength interval of between 3 μm and 15 μm is emitted from the IR device, whereas and wherein radiation below 3 μm is not emitted from the IR device and wherein no visible light is emitted from the IR device.

20. The light emitting device according to claim 1, wherein said light emitting device is configured to emit UV light with a radiant flux that is less than 300 W.

21. (canceled)

22. The light emitting device according to claim 1 wherein the power of the light emitting device is in the interval of 5 W-300 W.

23. An infrared (IR) heat emitting device for use in a pig creep for heating animals in the creep, comprising at least one thermal infrared source, and wherein the IR heat emitting device is configured such that radiation in a wavelength interval of between 3 μm and 15 μm is emitted from the IR device, and wherein radiation below 3 μm is not emitted from the IR device and wherein no visible light is emitted from the IR device.

24. (canceled)

25. The IR heat emitting device according to claim 23, comprising at least one screen covering the at least one IR source, the screen optionally (i) being transparent for some or all light wavelengths above 1000 nm, (ii) the screen absorbs light wavelengths below 1000 nm, (iii) wherein the screen is made from a material absorbing at least some light having wavelengths within the spectrum of visible light, or any two or more of (i) to (iii).

26. (canceled)

27. (canceled)

28. (canceled)

29. (canceled)

30. (canceled)

31. An animal nest, for enhancing the natural formation of ND3 in animals in the nest, comprising at least one light emitting device according to claim 1, wherein the at least one light emitting device is installed in the animal nest, such that light from the light emitting device is illuminating the inside of the animal nest.

32. (canceled)

33. (canceled)

34. (canceled)

35. (canceled)

36. (canceled)

37. A method for increasing the formation of ND3 in an animal by illuminating said animal with UVB light 24 hours a day using a light emitting device according to claim 1, which light emitting device is not emitting UV light below 285 nm and not emitting visible light between 380 and 750 nm, but is emitting light in a wavelength interval 290 nm-315 nm, the at least one light source optionally being situated no more than 1 meter away from the animal.

38. (canceled)

39. (canceled)

40. (canceled)

41. The method according to claim 37, wherein, during a day-time period, said animal is exposed to light with wavelength(s) in the interval 290 nm-315 nm simultaneously with light of other wavelengths.

42. (canceled)

43. (canceled)
